(19) United States
(12) Patent Application Publication
(10) Pub. No.: US 2020/0038422 A1
(43) Pub. Date: Feb. 6, 2020

(54) A COMPOSITION WITH MOBILITY BENEFITS IN AGEING, HEALTHY SUBJECTS

(71) Applicant: SOCIETE DES PRODUITS NESTLE S.A., Vevey (CH)

(72) Inventors: Marie Noelle Horcajada, Echenevex (FR); Jan Biehl, Thun (CH); Liya Denney, Cambridge (GB); Laurence Donato-Capel, Poliez-Ie-Grand (CH); Benoit Idieder, Villars sur Glane (CH)

(21) Appl. No.: 16/603,308

(22) PCT Filed: Apr. 12, 2018
(86) PCT No.: PCT/EP2018/059440

§ 371 Date: Oct. 7, 2019

(30) Foreign Application Priority Data
Apr. 13, 2017 (EP) 17166592.0

(51) Int. Cl. A61K 31/7008; A61K 33/06; A61K 31/375; A61K 31/593; A61K 33/30; A61K 35/20; A61K 9/00; A61P 19/02; A23C 9/158; A23C 9/152; A23C 9/18

(52) U.S. Cl. CPC A61K 31/7008; A61K 33/06; A61K 31/375; A61K 31/593; A61K 33/30; A23C 9/18; A61K 9/0095; A61P 19/02; A23C 9/158; A23C 9/1522; A61K 35/20

(57) ABSTRACT

The invention provides a composition useful in maintaining or improving mobility, preferably joint function and muscle strength in ageing, healthy subjects. The composition includes specific amounts of glucosamine or a pharmaceutically-acceptable derivative thereof, calcium, vitamin C, vitamin D and zinc. Also provided is the composition in the form of a powdered food product, and a beverage product comprising the composition in the form of a powder which has been reconstituted in a liquid. Also provided is the use of the composition or food or beverage product in maintaining or improving mobility, preferably joint function and muscle strength in ageing, healthy subjects.

### FIELD OF THE INVENTION

The present invention relates to a composition which is useful in maintaining or improving mobility in ageing, healthy subjects. In particular, the composition of the invention is useful in maintaining or improving joint function and muscle strength in ageing, healthy subjects. Additionally the invention relates to the composition in the form of a powdered food product, or in the form of a beverage product such as a milk drink.

### BACKGROUND TO THE INVENTION

Mobility refers to the ability of an individual to move their body freely and easily. It is typically assessed either with standardized performance-based tests or with reports of difficulty in carrying out specific tasks, particularly physical activities such as walking. Given that walking is heavily dependent on the functioning of the musculoskeletal system, it is no surprise that mobility declines with increasing age. Studies have shown that mobility is in fact amongst the most frequently cited health problems of ageing individuals. Mobility issues include joint pain, weight gain and lack of joint flexibility, where joint pain and flexibility have the highest impact on quality of life.

Joint flexibility is associated with joint functionality and muscle strength, that is to say the ability of a joint to be moved through its range of motion, bear weight and perform work, and the strength of the muscle supporting the joint. The range of motion of a joint will depend on the characteristics of the individual joint, as well as the muscles and ligaments supporting the joint, the capsule and the anatomy of the articulating surfaces. The knee joint for example typically has a range of motion from 0 degrees of extension (completely straight knee) to 135 degrees of flexion (fully bent knee joint). This is shown in FIG. 1.

A change in joint functionality can arise from changes in the connective tissues and/or changes in the bone. For example, connective tissue can increase in stiffness and/or decrease in strength because of age, and subchondral bone (the layer directly below the articular cartilage) can undergo a reduction in thickness and density. Unfortunately therefore, age-related loss of joint functionality will occur in all individuals.

Muscle strength also inevitably deteriorates with age. Keller et al., Muscles Ligaments Tendons J. 2013 October-December; 3(4); 346-350 reports on the strength and muscle mass loss with the ageing process and notes that muscle strength declines from people aged below 40 years to those aged above 40 years between 16.6% and 40.9%.

Glucosamine is widely used as a nutritional supplement for joint discomfort, especially in osteoarthritic subjects. Ng et al., Arthritis Research & Therapy, 2010, 12:R25 explains how glucosamine sulphate (GS) is believed to assist with building and repair of cartilage and can be used in combination with or without exercise by people with early hip or knee osteoarthritis.

Ng et al also includes a feasibility study on the combined effects of a progressive walking programme and GS intake on symptoms of osteoarthritis, and physical activity participation in people with hip or knee osteoarthritis. The study involved participants taking GS alone (1500 mg/day) for 6 weeks and then combining with a 12-week progressive walking programme at week 6. Ng et al was, however, focussed on subjects aged 40 to 75 years having physician-diagnosed osteoarthritis in at least one hip or knee.

Glucosamine is also included in Anlene® Total, a product marketed as a high calcium, low-fat milk powder which helps protect bone and joint health. Anlene® Total includes glucosamine hydrochloride together with calcium, vitamin D, magnesium, zinc and protein, and two glasses of the powder as a milk drink are said to provide a daily dose of 500 mg glucosamine and 1200 mg calcium.

DE 20 2016 003 259 U1 discloses a composition comprising glucosamine sulphate, organic potassium, calcium and magnesium salts, zing, vitamins C, D and B1 with berries and rose hip. CN 101366730 A discloses capsules comprising 1 to 70% glucosamine and 30 to 99% gelatine derived from donkey-hide. US 2008/0138417 A1 discloses a topical composition for use in treating various medical conditions. The composition includes a source of calcium, a source of potassium, a source of ascorbic acid, a source of lysine and a source of glutathione as anti-inflammatory components, a source of curcumin as an anti-oxidant, and an isoflavone. US 2008/0069862 A1 discloses a pet supplement which includes a joint preserving and a joint rebuilding composition comprising chicken collagen type II, glucosamine hydrochloride and chondroitin sulphate, vitamins C, D and K, minerals, and herbal extracts.

Warburton et al. (CMAJ 2006, 174(6):801-9) review the health benefits of physical activity (DOI:10.1503/cmaj.051351), in particular the role physical inactivity plays in the development of chronic disease and premature death.

There still, however, remains a need for compositions and food or beverage products that provide mobility benefits in ageing, healthy subjects. In particular, there remains a need for compositions and methods that maintain or improve joint function and muscle strength in ageing, healthy subjects.

### SUMMARY OF THE INVENTION

The present inventors have surprisingly found that a composition containing a specific amount of glucosamine together with specific amounts of calcium, vitamin C, vitamin D and zinc, provides mobility benefits in ageing, healthy subjects.

Whilst the use of glucosamine as a supplement for joint health improvement is known, the focus of previous work has been mainly on treating osteoarthritis. The present invention is not, however, concerned with osteoarthritic subjects. Instead, the present invention is focussed on ageing, healthy subjects. As will be discussed below, a “healthy” subject in the context of the present invention means a subject who has not been diagnosed by a physician as having a musculoskeletal disorder, preferably a subject who has not been diagnosed by a physician as having osteoarthritis.

The composition of the invention advantageously provides a twice daily nutritional milk-based drink that delivers an efficient dose of glucosamine. In particular the composition can deliver about 1500 mg of glucosamine, about 35 mg of vitamin C, about 850 mg of calcium, about 8 μg of vitamin D, about 6.5 mg zinc and about 11 g protein, daily. This mix of nutrients is believed to be beneficial for the joints, bones and muscles by preventing bone and muscle loss and alleviating joint discomfort (e.g. pain, stiffness etc.).

Additionally the present inventors have found that the composition of the invention further improves mobility when combined with a physical activity programme. This programme can be tailored to the needs of the subject, and is believed to have an important, positive effect on muscle function, mass strength along with bone and joint metabolism.

Accordingly, in one aspect the present invention provides a composition comprising (a) about 1500 mg to about 9000 mg of glucosamine or a pharmaceutically acceptable derivative thereof per 100 g of the composition, (b) about 300 mg to about 2000 mg of calcium per 100 g of the composition, (c) about 50 mg to about 150 mg of vitamin C per 100 g of the composition, (d) about 3 μg to about 30 μg of vitamin D per 100 g of the composition, and (e) about 5 mg to about 25 mg of zinc per 100 g of the composition.

In one embodiment the composition comprises (a) about 2500 mg to about 8000 mg of glucosamine or a pharmaceutically acceptable derivative thereof per 100 g of the composition, (b) about 300 mg to about 1700 mg of calcium per 100 g of the composition, (c) about 70 mg to about 110 mg of vitamin C per 100 g of the composition, (d) about 5 μg to about 16 μg of vitamin D per 100 g of the composition, and (e) about 10 mg to about 20 mg of zinc per 100 g of the composition.

In another embodiment the composition comprises (a) about 1500 mg to about 9000 mg of glucosamine or a pharmaceutically acceptable derivative thereof per 100 g of the composition, (b) about 300 mg to about 1700 mg of calcium per 100 g of the composition, (c) about 70 mg to about 110 mg of vitamin C per 100 g of the composition, (d) about 5 μg to about 16 μg of vitamin D per 100 g of the composition, and (e) about 10 mg to about 20 mg of zinc per 100 g of the composition.

In another embodiment the composition comprises (a) about 3000 mg to about 7500 mg of glucosamine or a pharmaceutically acceptable derivative thereof per 100 g of the composition, (b) about 1300 mg to about 2000 mg of calcium per 100 g of the composition, (c) about 70 mg to about 110 mg of vitamin C per 100 g of the composition, (d) about 7.5 μg to about 16 μg of vitamin D per 100 g of the composition, and (e) about 13 mg to about 20 mg of zinc per 100 g of the composition.

In another embodiment the composition comprises (a) about 2500 mg to about 8000 mg of glucosamine or a pharmaceutically acceptable derivative thereof per 100 g of the composition, (b) about 1350 mg to about 1700 mg of calcium per 100 g of the composition, (c) about 50 mg to about 150 mg of vitamin C per 100 g of the composition, (d) about 7.5 μg to about 16 μg of vitamin D per 100 g of the composition, and (e) about 13 mg to about 20 mg of zinc per 100 g of the composition.

In another embodiment the composition comprises (a) about 3000 mg to about 7500 mg of glucosamine or a pharmaceutically acceptable derivative thereof per 100 g of the composition, (b) about 1350 mg to about 1700 mg of calcium per 100 g of the composition, (c) about 70 mg to about 110 mg of vitamin C per 100 g of the composition, (d) about 5 μg to about 30 μg of vitamin D per 100 g of the composition, and (e) about 13 mg to about 20 mg of zinc per 100 g of the composition.

In another embodiment the composition comprises (a) about 3000 mg to about 7500 mg of glucosamine or a pharmaceutically acceptable derivative thereof per 100 g of the composition, (b) about 1350 mg to about 1700 mg of calcium per 100 g of the composition, (c) about 70 mg to about 110 mg of vitamin C per 100 g of the composition, (d) about 5 μg to about 30 μg of vitamin D per 100 g of the composition, and (e) about 10 mg to about 25 mg of zinc per 100 g of the composition.

In a preferred embodiment, the composition further comprises about 15 g to about 30 g of protein per 100 g of the composition, more preferably about 18 to about 24 g of protein per 100 g of the composition.

In another preferred embodiment the vitamin D is vitamin D₃.

In a further preferred embodiment the glucosamine is a glucosamine salt such as glucosamine sulphate.

In one embodiment, the composition comprises (a) about 3000 mg of glucosamine sulphate per 100 g of the composition, (b) about 1700 mg of calcium per 100 g of the composition, (c) about 70 mg of vitamin C per 100 g of the composition, (d) about 16 μg of vitamin D₃ per 100 g of the composition, (e) about 13 mg of zinc per 100 g of the composition, and (f) about 22 g of protein per 100 g of the composition.

Alternatively the composition comprises (a) about 7500 mg of glucosamine sulphate per 100 g of the composition, (b) about 1350 mg of calcium per 100 g of the composition, (c) about 110 mg of vitamin C per 100 g of the composition, (d) about 7.5 μg of vitamin D₃ per 100 g of the composition, and (e) about 20 mg of zinc per 100 g of the composition.

In one embodiment the composition provides about 200 to about 500 Kcal per 100 g of the composition, preferably about 406 Kcal/100 g; about 15 g to about 30 g of protein per 100 g of the composition, preferably about 22 g/100 g; about 5 g to about 25 g of fat per 100 g of the composition, preferably about 12 g/100 g; and about 40 g to about 60 g of carbohydrate per 100 g of the composition, preferably about 53 g/100 g.

In one embodiment the composition further provides about 200 mg to about 400 mg of sodium per 100 g of the composition, preferably about 360 mg/100 g.

In a preferred embodiment the composition is in the form of a powder, preferably suitable for reconstitution in a liquid, e.g. in water.

In another preferred embodiment the composition includes a milk powder, preferably a spray-dried milk powder.

Without wishing to be bound by theory, the inventors believe that the milk powder forms a matrix to support the nutrient mix. Additionally the milk powder contains components which contribute to the advantageous effect of the composition. For example, the milk powder includes calcium and protein.

Calcium plays a key role in providing bone structure and bone strength, whilst protein is useful for muscles, bones and joints. The amount of calcium in the composition of the invention can therefore refer to calcium which is added as a separate component, e.g. calcium carbonate or another acceptable form of calcium, and/or calcium which is present in the milk powder. The present invention is not limited in this respect.

When the composition includes a milk powder, the amount of calcium in the composition is preferably about 900 mg to about 2000 mg per 100 g of the composition, more preferably about 1200 mg to about 1800 mg per 100 g of the composition.

In one embodiment the composition is in the form of a powdered food product.

In another embodiment the composition is in the form of a beverage product. The beverage product is preferably the composition in the form of a powder which has been reconstituted with a liquid. Preferably the liquid is water. When the composition includes a milk powder, the beverage product may be a milk drink.

In another aspect, the present invention provides a composition as described herein or a beverage product described herein for use in maintaining or improving mobility in an ageing, healthy subject.

In another aspect, the present invention provides a composition as described herein or a beverage product described herein for use in maintaining or improving joint function and muscle strength in an ageing, healthy subject.

The ageing, healthy subject may, for example, be a human subject over the age of 30, 35, 40, 45, 50, 55, 60, 65, 70, 75, 80, 85, 90, 95 or 100 years old. In a preferred embodiment the ageing, healthy subject is aged over about 40 years old, e.g. between about 40 and about 75 years old.

In another preferred embodiment the subject has not been physician-diagnosed with a musculoskeletal disorder, more preferably the subject has not been physician-diagnosed with osteoarthritis. In one embodiment this subject is a pre-osteoarthritis subject. The term “pre-osteoarthritis” is discussed in more detail below.

In one embodiment the subject is aged over about 40 years old and is a pre-osteoarthritis subject.

In one embodiment the composition is administered orally twice a day.

In one embodiment the use further comprises at least one physical activity, preferably physical activity is carried out 2 to 4 times a week, more preferably 3 times a week.

In a preferred embodiment the physical activity comprises at least 130 minutes of walking per week.

In another aspect the invention provides a method of maintaining or improving mobility in an ageing, healthy subject, wherein said method comprises administering to the subject a composition or beverage product of the present invention.

In another aspect the invention provides a method of maintaining or improving joint function and muscle strength in an ageing, healthy subject, wherein said method comprises administering to the subject a composition or beverage product of the present invention.

In another aspect the invention provides the use of a composition of the present invention or a beverage product of the present invention for the manufacture of a medicament for maintaining or improving mobility in an ageing, healthy subject.

In another aspect the invention provides the use of a composition of the present invention or a beverage product of the present invention for the manufacture of a medicament for maintaining or improving joint function and muscle strength in an ageing, healthy subject.

In another aspect the invention provides the use of a composition of the present invention or a beverage product of the present invention for maintaining or improving mobility in an ageing, healthy subject.

In another aspect the invention provides the use of a composition of the present invention or a beverage product of the present invention for maintaining or improving joint function and muscle strength in an ageing, healthy subject.

### DESCRIPTION OF THE DRAWINGS

FIG. 1 is a schematic representation of the typical range of motion for a knee joint. The range of motion is typically from 0 degrees of extension (completely straight knee) to 135 degrees of flexion (fully bent knee joint).

FIG. 2 compares the walking distance (in metres) achieved in the six minute walk test (6MWD) of Example 2 for low, median and high subjects at the baseline, after the nutrition period (V3) and at the end of the study (V7).

FIG. 3 compares the torque max flexor (nM) achieved in the dominant leg strength testing of Example 2 for low, median and high subjects at the baseline and at the end of the study (V7).

FIG. 4 compares the total work flexor (J) achieved in the dominant leg strength testing of Example 2 for low, median and high subjects at the baseline and at the end of the study (V7).

FIG. 5 shows the results of the balance outcome test in Example 2. This figure is a plot of balancing time (in minutes) against visit number (V1, V3, V4, V6 and V7) for both the left leg (O) and the right leg (Δ).

FIG. 6 compares the EQ5D-5L scores to assess quality of life at baseline, visit 3 (end of nutrition period) and visit 7 (end of study).

### DETAILED DESCRIPTION OF THE INVENTION

Various preferred features and embodiments of the present invention will now be described by way of non-limiting examples. It is noted that the various aspects, features, examples and embodiments described in the present application may be compatible and/or combined together. In particular, features, examples and embodiments described in the context of the composition of the invention are equally applicable to the uses and methods of the invention.

Unless otherwise indicated, all amounts indicated for nutrients are expressed as amounts per 100 g of the composition. For example, “about 2500 mg/100 g of glucosamine sulphate” means about 2500 mg glucosamine sulphate per 100 g of the composition.

Ageing, Healthy Subject

The subjects referred to in the present disclosure as the target of the compositions are ageing, healthy subjects. Preferably these subjects are human.

By “ageing” is meant that the subject is over the age of 30, 35, 40, 45, 50, 55, 60, 65, 70, 75, 80, 85, 90, 95 or 100 years old. Preferably the subject is over about 40, more preferably over about 50, even more preferably over about 60 years old.

In one embodiment the subject is aged between about 40 and about 70 years, preferably between about 45 and about 70 years, more preferably between about 45 and about 65 years.

By “healthy is meant that the subject has not been physician-diagnosed with a musculoskeletal disorder. Musculoskeletal disorders (MSDs) are conditions that can affect your muscles, bones and joints. They are also conditions which have a higher risk of developing with age.

Non-limiting examples of musculoskeletal disorders include tendinitis, epicondylitis, carpal tunnel syndrome, osteoarthritis, rheumatoid arthritis, fibromyalgia, bone fractures, osteoporosis, and gout. In one embodiment the subject has not been diagnosed by a physician as having osteoarthritis.

In one embodiment the subject may experience ill-effects associated with ageing. Non-limiting examples of such ill-effects include joint discomfort, joint stiffness, joint pain, muscular pain, vision problems, mobility issues, cognitive issues, lack of bone density, cardiovascular risks, bone pain, high blood fat/cholesterol, lack of energy/fatigue, urinary issues, high blood sugar and weight gain.

In one embodiment the subject may suffer from one or more ill-effects associated with the joints such as joint discomfort, joint stiffness or joint pain, preferably the subject may suffer from joint discomfort, e.g. knee, hand (including finger and thumb), and/or hip joint(s) discomfort.

In a preferred embodiment, “ageing, healthy subject” refers to a female or male human subject aged over about 40 old, who has not been diagnosed by a physician as having osteoarthritis. In a particularly preferred embodiment, the subject suffers from one or more of joint discomfort, joint stiffness or joint pain, preferably one or more of joint discomfort or joint pain, more preferably joint discomfort.

These subjects are referred to herein as pre-osteoarthritis subjects. They are subjects which have not been physician-diagnosed with osteoarthritis but show at least one ill-effect associated with a joint, e.g. the knee joint, the hand joints or the hip joint.

Glucosamine

The skilled person will have a detailed and thorough understanding of the use of glucosamine in nutritional compositions. Briefly, glucosamine is an oral dietary supplement which is marketed to support the structure and function of joints.

The compositions of the invention include glucosamine or a pharmaceutically acceptable derivative thereof. By derivative is meant any pharmaceutically acceptable salt, solvate and/or reaction product thereof which does not affect the activity of glucosamine. In one embodiment glucosamine or a pharmaceutically acceptable derivative thereof means glucosamine, glucosamine sulphate, glucosamine hydrochloride or N-acetylglucosamine. N-acetylglucosamine is for instance an amide formed by the reaction of glucosamine and acetic acid. Preferably the composition includes glucosamine sulphate.

The composition includes about 1500 mg to about 9000 mg of glucosamine or a pharmaceutically acceptable derivative thereof per 100 g of the composition.

In one embodiment the composition includes about 1500 mg to about 8000 mg, about 2000 mg to about 8000 mg, about 2500 mg to about 8000 mg, about 2500 mg to about 7500 mg, about 3000 mg to about 7500 mg, about 3000 mg to about 7000 mg, about 3500 mg to about 6000 mg, or about 4000 to about 6000 mg of glucosamine or a pharmaceutically acceptable derivative thereof per 100 g of the composition.

In a preferred embodiment the composition includes about 1500 mg to about 8000 mg of glucosamine or a pharmaceutically acceptable derivative thereof per 100 g of the composition. For example about 1500 mg or about 2000 mg or about 2500 mg or about 3000 mg or about 3500 mg or about 4000 mg or about 4500 mg or about 5000 mg or about 5500 or about 6000 mg or about 6500 mg or about 7000 mg or about 7500 mg or about 8000 mg of glucosamine or a pharmaceutically acceptable derivative thereof per 100 g of the composition.

In one embodiment the composition includes about 2500 mg to about 8000 mg of glucosamine or a pharmaceutically acceptable derivative thereof per 100 g of the composition. In one embodiment the composition includes about 3000 mg of glucosamine or a pharmaceutically acceptable derivative thereof per 100 g of the composition.

In another embodiment the composition includes about 7500 mg of glucosamine or a pharmaceutically acceptable derivative thereof per 100 g of the composition.

Another way of defining the composition is that glucosamine or a pharmaceutically acceptable derivative thereof is comprised therein in an amount such that the total daily intake derived from the composition of the invention is about 750 mg to about 3000 mg, preferably about 1000 mg to about 2500 mg, most preferably about 1500 mg.

Preferably, the pharmaceutically acceptable derivative of glucosamine is glucosamine sulphate.

Calcium

Calcium may be present in the composition of the invention as a component of the milk powder and/or as an independent component.

Calcium may for example be incorporated in the composition of the invention as such or in the form of a physiologically acceptable salt and/or via any source comprising calcium. For example calcium carbonate, calcium chloride, calcium salts of citric acid, calcium gluconate, calcium glycerophosphate, calcium lactate, calcium hydroxide, or calcium salts of orthophosphoric acid may be used.

Preferably calcium is at least incorporated in the composition of the invention in the form of calcium carbonate.

More preferably calcium is incorporated in the composition of the invention as part of the milk powder and independently in the form of calcium carbonate.

The composition includes about 300 mg to about 2000 mg of calcium per 100 g of the composition. When the composition includes a milk powder the composition may include about 900 mg to about 2000 mg of calcium per 100 g of the composition.

In one embodiment the composition includes about 300 mg to about 1700 mg, about 400 mg to about 1600 mg, about 500 mg to about 1500 mg, about 600 mg to about 1500 mg, about 700 mg to about 1500 mg, about 800 mg to about 1500 mg, about 900 mg to about 1450 mg, about 1000 mg to about 1400 mg, about 1100 mg to about 1300 mg of calcium per 100 g of the composition.

In one embodiment the composition includes about 1300 mg to about 1700 mg, about 1350 mg to about 1650 mg, about 1400 mg to about 1650 mg, or about 1400 mg to about 1600 mg of calcium per 100 g of the composition.

In a preferred embodiment the composition includes about 1350 mg to about 1700 mg of calcium per 100 g of the composition. For example about 1350 mg or about 1375 mg or about 1400 mg or about 1425 mg or about 1450 mg or about 1475 or about 1500 mg or about 1525 mg or about 1550 mg or about 1575 mg or about 1600 mg or about 1625 mg or about 1650 mg or about 1675 mg or about 1700 mg of calcium per 100 g of the composition.

In one embodiment the composition includes about 1700 mg of calcium per 100 g of the composition.

In another embodiment the composition includes about 1350 mg of calcium per 100 g of the composition.

Alternatively, calcium is comprised in the composition in an amount such that the total daily intake derived from the composition of the invention is from about 100 to about 2500 mg, more preferably from about 200 to about 2000 mg, more preferably, from about 250 to about 1500 mg, even more preferably from about 500 to about 1000 mg.

In a preferred embodiment, the composition according to the present invention comprises levels of calcium such that the total daily intake derived from the composition of the invention is about 850 mg.

Vitamin D

In one embodiment the composition includes vitamin D in the form of vitamin D₃, also known as cholecalciferol. In another embodiment the composition includes vitamin D in the form of vitamin D₂, also known as ergocalciferol.

Preferably the vitamin D is vitamin D₃.

The composition includes about 3 μg to about 30 μg of vitamin D per 100 g of the composition.

In one embodiment the composition includes about 5 μg to about 25 μg, about 5 μg to about 22.5 μg, about 5 μg to about 16 μg, about 7.5 μg to about 22.5 μg, about 7.5 μg to about 20 μg, about 7.5 μg to about 17.5 μg, about 7.5 μg to about 16 μg, about 7.5 μg to about 15 μg, about 10 μg to about 12.5 μg of vitamin D per 100 g of the composition.

In a preferred embodiment the composition includes about 5 μg to about 16 μg per 100 g of dry composition. For example about 5 μg or about 5.5 μg or about 6 μg or about 7 μg or about 7.5 μg or about 8 μg or about 8.5 μg or about 9 μg or about 9.5 μg or about 10 μg or about 10.5 μg or about 11 μg or about 11.5 μg or about 12 μg or about 12.5 μg or about 13 μg or about 13.5 μg or about 14 μg or about 14.5 μg or about 15 μg or about 15.5 μg or about 16 μg of vitamin D per 100 g of the composition.

In one embodiment the composition includes about 16 μg of vitamin D per 100 g of the composition.

In another embodiment the composition includes about 7.5 μg of vitamin D per 100 g of the composition.

Alternatively the composition comprises an amount of vitamin D such that the total daily intake derived from the composition of the invention is from about 0.2 to about 30 μg, more preferably from about 0.5 to about 25 μg, more preferably from about 0.5 to about 15 μg, even more preferably from about 1 to about 10 μg, more preferably still, from about 2 to about 8 μg.

In one highly preferred embodiment, the composition comprises an amount of vitamin D such that the total daily intake derived from the composition of the invention is about 8 μg.

Vitamin C

The composition includes about 50 mg to about 150 mg of vitamin C per 100 g of the composition.

In one embodiment the composition includes about 50 mg to about 125 mg, about 55 mg to about 125 mg, about 55 mg to about 120 mg, about 60 mg to about 120 mg, about 60 mg to about 115 mg, about 65 mg to about 115 mg, about 65 mg to about 110 mg, about 70 mg to about 110 mg, about 70 mg to about 100 mg, about 75 mg to about 100 mg, about 75 mg to about 95 mg, or about 80 mg to about 95 mg of vitamin C per 100 g of the composition.

In a preferred embodiment the composition includes about 70 mg to about 110 mg of vitamin C per 100 g of dry composition. For example about 70 mg or about 75 mg or about 80 mg or about 85 mg or about 90 mg or about 95 mg or about 100 mg or about 105 mg or about 110 mg of vitamin C per 100 g of the composition.

In one embodiment the composition includes about 70 mg of vitamin C per 100 g of the composition.

In another embodiment the composition includes about 110 mg of vitamin C per 100 g of the composition.

Alternatively the composition comprises an amount of vitamin C such that the total daily intake derived from the composition of the invention is from about 5 to about 50 mg, more preferably from about 10 to about 45 mg, more preferably from about 15 to about 40 mg, even more preferably from about 20 to about 40 mg.

In one highly preferred embodiment, the composition comprises an amount of vitamin C such that the total daily intake derived from the composition of the invention is about 35 mg.

Zinc

Zinc may be incorporated in the compositions of the invention in the form of a physiologically acceptable salt such as for example: zinc nitrate, zinc sulfate, zinc gluconate, zinc acetate or mixtures thereof, or in the form of a physiologically acceptable zinc complex (such as for example zinc picolinate) or mixtures thereof.

The composition includes about 5 mg to about 25 mg of zinc per 100 g of the composition.

In one embodiment the composition includes about 8 mg to about 25 mg, about 10 mg to about 25 mg, about 11 mg to about 25 mg, about 11 mg to about 24 mg, about 12 mg to about 23 mg, about 12 mg to about 22 mg, about 13 mg to about 21 mg, about 13 mg to about 20 mg, about 14 mg to about 19 mg or about 15 mg to about 18 mg of zinc per 100 g of the composition.

In a preferred embodiment the composition includes about 10 mg to about 20 mg of zinc per 100 g of the composition. For example about 10 mg or about 11 mg or about 12 mg or about 13 mg or about 14 mg or about 15 mg or about 16 mg or about 17 mg or about 18 mg or about 19 mg or about 20 mg of zinc per 100 g of the composition.

In one embodiment the composition includes about 13 mg of zinc per 100 g of the composition.

In another embodiment the composition includes about 20 mg of zinc per 100 g of the composition.

Alternatively the composition comprises an amount of zinc such that the total daily intake derived from the composition of the invention is from about 1 to about 25 mg, more preferably from about 2 to about 20 mg, more preferably from about 4 to about 10 mg, even more preferably from about 5 to about 8 mg.

In one highly preferred embodiment, the zinc is comprised in the composition in an amount such that the total daily intake derived from the composition is about 6.5 mg.

Protein

In one embodiment the composition of the invention includes protein.

The term “protein” refers to polymers of amino acids, and includes polypeptides and peptides. The term “protein” does not encompass free amino acids, which may also be present in the composition of the invention.

The protein may be included at an amount of about 15 g to about 30 g per 100 g of the composition. In one embodiment the protein is included at an amount of about 16 to about 25 g or about 17 to about 24 g or about 18 to about 23 g or about 20 to 23 g of protein per 100 g of the composition.

In a preferred embodiment the composition includes about 22 g of protein per 100 g of the composition.

Any suitable dietary protein may be used for example animal proteins (such as milk proteins, meat proteins and egg proteins); vegetable proteins (such as soy protein, wheat protein, rice protein, and pea protein); or combinations thereof.

Non-limiting examples of proteins include casein, alpha-lactalbumin, whey, soy protein, rice protein, corn protein, oat protein, barley protein, wheat protein, rye protein, pea protein, egg protein, sunflower seed protein, potato protein, fish protein, meat protein, lactoferrin, serum albumin, immunoglobins, and combinations thereof.

Particularly preferred are milk proteins such as casein and whey, and soy proteins. Milk proteins, such as casein and whey, are the most preferred.

In one embodiment the protein is present when the composition comprises a milk powder.

Milk Powder

In one embodiment the composition of the invention comprises a milk powder.

Any milk powder known in the art can be included, provided that the overall composition meets the content specification of the invention. Non-limiting examples of the milk powder include skimmed milk powder, whole milk powder, non-fat dry milk, yoghurt powder and reduced fat milk powder.

In one embodiment the milk powder is a standardised milk powder prepared by spray-drying solid non-fat, milk fat, a bulking agent and an emulsifier. The bulking agent may be maltodextrin, e.g. maltodextrin DE 37-41, and the emulsifier may be lecithin.

Additional Components

The composition of the invention may include additional components or excipients known to be employed in the type of composition in question. Such additional components will be familiar to the skilled person in the art.

The composition may for example comprise additional minerals, vitamins, salts, functional additives including, for example, palatants, colorants, emulsifiers, antimicrobial or other preservatives. Minerals that may be useful in such compositions include, for example, phosphorous, potassium, sodium, iron, chloride, boron, copper, magnesium, manganese, iodine, selenium, chromium, molybdenum, fluoride and the like. Minerals are usually added in their salt form.

Examples of additional vitamins that may be useful in compositions described herein include water soluble vitamins (such as thiamin (vitamin B1), riboflavin (vitamin B2), niacin (vitamin B3), pantothenic acid (vitamin B5), pyridoxine (vitamin B6), biotin (vitamin B7), myo-inositol (vitamin B8), folic acid (vitamin B9), and cobalamin (vitamin B12)) and fat soluble vitamins (such as vitamin A, vitamin E, and vitamin K) including salts, esters or derivatives thereof. Inulin, taurine, carnitine, amino acids, enzymes, coenzymes, and the like may be useful to include in various embodiments.

The skilled person in the art can identify appropriate amounts of the above mentioned additional components based on, for example, the nature and purpose of the composition, the target subject and the dosage of the composition.

The composition may also contain minerals and micronutrients such as trace elements and vitamins in accordance with the recommendations of Government bodies such as the USRDA or the Chinese RDA. For example, the compositions may also contain one or more vitamins and minerals understood to be essential in the daily diet and in nutritionally significant amounts.

Preferably, the composition does not contain hydrocolloids, such as gelatine, modified starch, xanthan gum, carrageenan, or gellan gum.

Type of Composition

The composition may be any type of composition suitable for administration to an ageing, healthy human subject. Compositions of the invention are preferably in solid form. The composition may, for example, be in the form of a tablet, dragée, capsule, gel cap, powder, granule, solution, emulsion, suspension, coated particle, spray-dried particle or pill.

In another embodiment the composition may be in the form of a powder. The powder may, for example, be a spray-dried powder, a freeze-dried powder or a dry-mixed powder.

A powder composition may be contained in a sachet or a container.

In one embodiment the powder composition is contained in a multi-serving container.

In another embodiment the powder composition is contained in a sachet in an amount suitable for one serving.

The term “serving” refers to a total amount of composition which can deliver about 750 mg of glucosamine, about 17.5 mg of vitamin C, about 425 mg of calcium, about 4 μg of vitamin D, about 3.25 mg zinc and about 5.5 g protein. In one embodiment a serving is about 20 g to about 50 g, preferably about 20 g to about 30 g, most preferably about 25 g of the composition.

A powder composition according to the present invention may be used to sprinkle onto a food or beverage. More preferably the composition is in the form of a powder for reconstituting in a liquid.

A particularly preferred embodiment provides a composition according to the invention in the form of a sachet containing a powder, wherein the powder can be dispersed or dissolved into a beverage (e.g. water, fruit juice, milk, etc.) to provide a palatable nutrient liquid for oral administration.

In one embodiment the composition is in the form of a powdered food product.

In another embodiment the composition is in the form of a beverage product, where the beverage comprises the composition in the form of a powder reconstituted with a liquid. Preferably the liquid is water.

Example food or beverage products include a dairy product, a dairy dessert, a milk drink, cream, ice cream mix, and soya product. When the composition comprises a milk powder, the beverage product may be a milk drink. This milk drink is preferably prepared by reconstituting the powder composition described herein in liquid, e.g. water.

Nutritional Composition

The composition may be in the form of a nutritional composition or a nutritional supplement.

The term “nutritional supplement” refers to a product which is intended to supplement the general diet of a subject.

The composition may be in the form of a complete nutritional product. The term “complete nutritional product” refers to a product which is capable of being the sole source of nourishment for the subject, for example including a source of protein, carbohydrate and fat.

In one preferred embodiment of the present invention, the composition comprises one or more of a protein source, a fat source and a carbohydrate source. Each of these sources may be any suitable source known in the art.

The carbohydrate content of the composition of the invention is preferably in the range about 30 to about 70 g per 100 g of the composition, more preferably about 40 to about 60 g per 100 of the composition. In a particular preferred embodiment the carbohydrate content of the composition is about 53 g per 100 g of the composition.

Example carbohydrates include lactose, saccharose, maltodextrin and starch. Mixtures of carbohydrates may be used. In one embodiment the carbohydrate comprises maltodextrin. In one embodiment, the composition comprises at least 5, 10, 15 or 20% of maltodextrin, wherein the % is based on the solids content of the composition.

In one embodiment the carbohydrates comprises lactose. In one embodiment the composition comprises at least 40%, 50%, 60% or 70% solids content of lactose.

In one embodiment the carbohydrate comprises lactose and maltodextrin.

The fat content of the composition of the invention is preferably in the range of about 5 to about 20 g per 100 g of the composition, more preferably in the range of about 10 to about 15 g per 100 g of the composition. In a particularly preferred embodiment the fat content of the composition is about 12 g per 100 g of the composition.

The fat may be any lipid or fat which is suitable for use in the composition. In one embodiment the fat comprises milk fat.

Mobility Benefits

The compositions, uses and methods of the invention provide mobility benefits. In particular, the compositions, uses and methods of the invention provide for the maintenance or improvement of joint function and muscle strength in an ageing, healthy subject.

The term “mobility” refers to the ability of an individual to move.

The maintenance or improvement in mobility can be measured by various methods known in the art and illustrated in the examples below.

Suitable methods for assessing mobility include gait speed, the 6 minute walking distance test, short physical performance battery and self-reported mobility. All of these methods are known in the art. The 6 minute walking test is for instance described in Macias-Hernandez, S. I. et al., Clinical Rheumatology, Volume 35, Issue 8, 1 Aug. 2016, Pages 2087-2092 which is incorporated herein by reference.

The term “joint functionality” or “joint function” refers to the ability of a joint to move in its full range of motion, bear weight and perform work.

As is known in the art, individual joints have a predetermined range of motion. This range of motion is commonly measured in degrees. For example, hips, knees, ankles, feet, joints of the feet, joints of the toe, shoulders, elbows, wrists, and hand and finger joints all have different ranges of motion, and there are generally accepted values for a normal range of motion in each of these joints. These values would be known to a skilled person in the art.

The maintenance or improvement in joint function and muscle strength can be measured by various methods known in the art and illustrated in the examples below.

A suitable method for assessing joint function and muscle strength is a dominant leg strength assessment using an isokinetic dynamometer. This assessment can, for example, include measuring isokinetic parameters such as torque max flexor, torque max extensor, total work flexor, total work extensor, and ratios thereof.

The term “maintain”, “maintains” or “maintaining” refers to a particular parameter, such as mobility, remaining substantially unchanged over a period of time (e.g. 5, 10, 15, 20, 25, 30, 40, 50 or more years).

In one embodiment, the mobility of a subject increases by at least about 1%, about 2%, about 3%, about 4%, about 5%, about 6%, about 10%, about 15% or about 20%.

In another embodiment, the mobility of a subject increases by about 1 to about 6%, about 1 to about 10% or about 1 to about 20%.

In a preferred embodiment the mobility of a subject increases by at least about 6% measured according to the 6 minute walking distance test (6MWD).

In one embodiment the joint function and muscle strength of a subject increases by at least about 1%, about 2%, about 3%, about 4%, about 5%, about 6%, about 10%, about 15% or about 20%.

In another embodiment, the joint function and muscle strength of a subject increases by about 1 to about 6%, about 1 to about 10% or about 1 to about 20%.

Preferably the muscle is skeletal muscle.

In one embodiment the increase in joint function and muscle strength is determined with an isokinetic dynamometer.

Physical Activity

In one embodiment the composition for use according to the invention includes at least one physical activity. By “physical activity” is meant any bodily movement produced by skeletal muscles that requires energy expenditure. This is the definition given by the World Health Organisation.

Non-limiting examples of physical activity include strength exercises, Tai-Chi, balance exercises, aerobic exercises such as walking, dancing, jogging, running, cycling or participating in sports, and flexibility exercises such as yoga, stretching and Pilates.

As explained hereinabove, the present invention advantageously provides a composition which can be combined with at least one physical activity to maintain or improve joint mobility. The physical activity can form part of an exercise programme which may be tailored to the needs of the subject in question.

In a preferred embodiment the at least one physical activity is carried out at least 2 to 4 times a week, most preferably at least 3 times a week. For example, Tai Chi may be carried out once a week together with strength exercises twice a week. Aerobic exercises may also be carried every day.

In another preferred embodiment the at least one physical activity comprises walking for at least 130 minutes per week.

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

There now follows a series of non-limiting examples that serve to illustrate the invention.

### EXAMPLES

### Example 1

Preparation of the composition, powdered food product and beverage product

A milk powder was prepared by spray drying, according to a method known in the art, the following mixture of components:

| Components | Percent (%, solid) |
|---|---|
| SNF (Solid Non Fat = lactose, proteins | 65.821 |
| and minerals from milk) | |
| Milk FAT | 11.544 |
| Maltodextrin DE (37-41) (=bulking agent) | 11.178 |
| Lecithin (emulsifier) | 0.321 |

The spray-dried milk powder was then dry-mixed with the following nutrients to form a powder:

| Components | Percent (%, solid) |
|---|---|
| Calcium carbonate | 2.150 |
| Glucosamine sulphate | 4.320 |
| Vitamin Mineral Premix | 0.950 |
| (including zinc, vitamin C and vitamin D₃) | |
| Flavour | 0.126 |
| Milk Powder | 0.090 |
| Water | 3.500 |
| TOTAL | 100.000 |

The nutritional analysis of the product was as follows:

| | | |
|---|---|---|
| Energy | Kcal/100 g | 406 |
| Protein | g/100 g | 22 |
| Fat | g/100 g | 12 |
| Carbohydrates | g/100 g | 53 |
| Na | mg/100 g | 360 |
| Vitamin D3 | μg/100 g | 16 |
| Vitamin C | mg/100 g | 70 |
| Ca | mg/100 g | 1700 |
| Zn | mg/100 g | 13 |
| Glucosamine/GS | mg/100 g | 3000 |
| | Serving Size | 25 g |

To prepare a beverage product according to the present invention, the 25 g of powder was reconstituted in approximately 180 ml of water.

When taken twice a day, this product provided 1500 mg of glucosamine sulphate, 6.5 mg of zinc, 850 mg of calcium, 35 mg of vitamin C, and 8 μg of vitamin D3, daily. The product also provided 203 Kcal, 11 g protein, 6 g fat, 26.5 g carbohydrates and 180 mg sodium, daily.

### Example 2

Combining nutritional supplementation and a physical activity programme in healthy 50 year+ volunteers with knee joint discomfort

The aim of this example was to evaluate the efficacy of a 6-month intervention comprising the composition of the invention and a physical activity programme on outcomes in ageing, healthy pre-osteoarthritis volunteers.

Materials and Methods

This example is based on a single-centre, single arm, baseline-controlled, open label, nutrition and exercise intervention clinical trial. A total of 54 healthy subjects with knee joint discomfort were recruited, aged 45-65 years, and each volunteer was provided with a composition according to the present invention to be taken orally twice a day for 6 months.

The composition provided about 1500 mg of glucosamine sulphate, about 850 mg of calcium, about 35 mg of vitamin C, about 8 μg of vitamin D (D₃), about 6.5 mg of zinc and about 11 g of protein, daily. The milk-based drink was prepared by pouring 180 ml of warm water into a container and stirring 25 g of the milk powder containing the nutrient mix therein. This preparation was repeated twice a day.

Initially the composition was taken alone, i.e. without physical activity, for 2 months. It was then combined with an exercise programme for 4 months. The physical activity/exercise programme included a combination of Tai Chi, strength exercises and aerobic exercises (walking), 3 times per week.

Daily step counts were recorded using a pedometer (Garmin).

The first primary outcome was mobility. This was assessed using a validated physical function test: the 6 minute walking distance test (6MWD).

The second primary outcome was joint function and muscle strength. This was assessed using another validated physical function test: a dominant leg strength assessment using an isokinetic dynamometer. This assessment involved measuring various isokinetic parameters (torque max flexor, total work flexor, torque max extensor, total work extensor, ratio of torque flexor/extensor) using the dynamometer.

The secondary outcomes were assessed with a KOOS score (Knee injury and Osteoarthritis Outcome Score), a Visual Analog Scale (VAS) score, a balance test, biomarkers for bone and joint metabolism and a quality of life score (evaluated by an EQSD-5L questionnaire).

Assessments were conducted at baseline (V1), at 1 month (V2), at 2 months after the nutrition only period (V3), and then every month for the remaining time of the study (V4 to V7). Subjects were classified into three levels based on their average daily step count:

low<=9999;10,000<=average<=13999; and high>=14000.

Baseline parameters were compared to 6 months of intervention using an Analysis of Covariance (ANCOVA) model with the baseline value as covariate and levels of step counts as fixed effect.

To support the main statistical analysis, a longitudinal analysis of change from baseline at each visit was performed using Mixed Model Repeated Measures (MMRM). Results were obtained for the expected change from baseline for outcomes at each visit for three possible baseline quartile (Q) values, i.e. Q1, Q2 as median, and Q3 of the outcome at baseline. A linear model was used for the analysis.

Results: Primary Outcomes

6 Minute Walking Distance Test (6MWD)

FIG. 2 shows the increase in walking distance for each sub-group of subjects at baseline, V3 (end of nutrition period) and V7 (end of the study). It can be seen from this figure that the walking distance significantly improved by approximately 6% in all subjects at the end of the study; the change from baseline to 6 months was significantly increased for each level of step count (p<0.001).

The supportive statistical analysis of the 6MWD results using Mixed Model Repeated Measures also showed that for Q1, the change from baseline at each visit was statistically significant following the nutrition period alone (p<0.05). In other words, the improvement in walking distance was observed after nutrition alone in the lowest scores at baseline.

Dominant Leg Strength

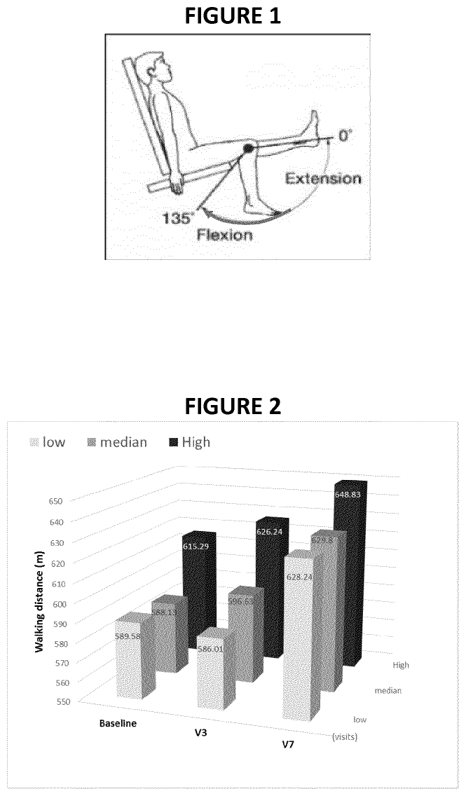

FIG. 1 shows the typical range of motion for the knee joint. “Flexion” refers to the strength to flex the leg and “extension” refers to the strength to extend the leg.

The isokinetic parameters determined to assess dominant leg strength (and hence joint function and muscle strength) were torque max flexor, total work flexor, torque max extensor, total work extensor and the ratio of torque flexor/extensor.

The change from baseline for each of these parameters at V7 (i.e. the end of the study) are shown in Table 1 below.

TABLE 1

| Isokinetic Parameter | | Estimate | Std. Error | 2.5% | 97.50% | P-value |
|---|---|---|---|---|---|---|
| Torque Max Extensor (Nm) | Low step count | 0.10 | 0.07 | −0.06 | 0.27 | 0.1535 |
| | Average step count | 0.05 | 0.07 | −0.12 | 0.22 | 0.5192 |
| | High step count | 0.06 | 0.10 | −0.17 | 0.29 | 0.5141 |
| Torque Max Flexor (Nm) | Low step count | 0.11 | 0.04 | 0.04 | 0.19 | 0.0042 |
| | Average step count | 0.06 | 0.04 | −0.02 | 0.13 | 0.1388 |
| | High step count | 0.12 | 0.05 | 0.02 | 0.23 | 0.0190 |
| Total Work Extensor (J) | Low step count | 0.49 | 0.40 | −0.32 | 1.30 | 0.2289 |
| | Average step count | 0.49 | 0.41 | −0.34 | 1.31 | 0.2434 |
| | High step count | 0.38 | 0.55 | −0.73 | 1.49 | 0.4896 |
| Total Work Flexor (J) | Low step count | 0.51 | 0.22 | 0.06 | 0.96 | 0.0278 |
| | Average step count | 0.26 | 0.23 | −0.20 | 0.73 | 0.2549 |
| | High step count | 0.57 | 0.31 | −0.05 | 1.19 | 0.0724 |
| Ratio Torque Flexor/Extensor | Low step count | 0.13 | 0.06 | −0.00 | 0.26 | 0.0584 |
| | Average step count | 0.06 | 0.07 | −0.07 | 0.20 | 0.3531 |
| | High step count | 0.17 | 0.09 | −0.01 | 0.35 | 0.0666 |

The ANCONA model will include baseline isokinetic strength parameter value as covariate and levels of Garmin compliance as fixed effect.
For ‘Ratio Torque flexor/extensor)’, natural log of the ratio is taken and then change is calculated for log transformed values due to non-normality of the data.
Analysis for Torque max extensor is performed at 2.5% level of significance and for rest of the parameters significance level is 5%.

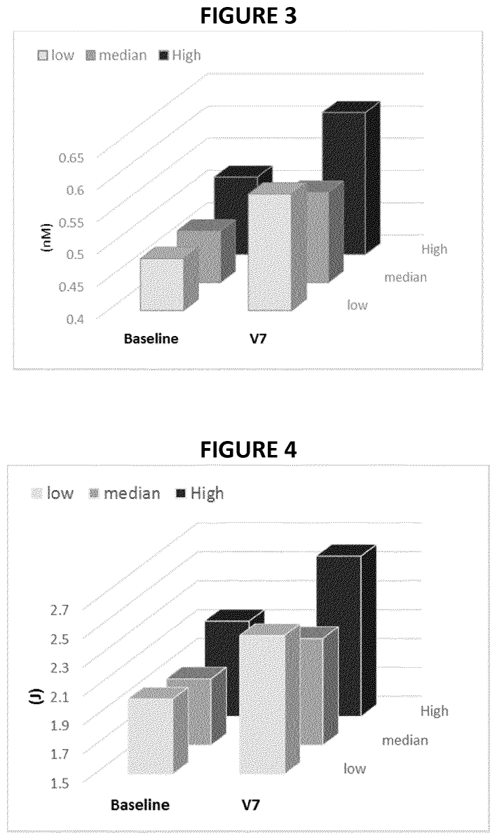

FIG. 3 plots the mean values (nM) for the torque max flexor at baseline and at the end of the study (V7) for each step-count (all values were divided by the subject's weight (in kg) at each visit). These values, together with the results in Table 1 above, show that flexor maximum torque was significantly increased following the 6-month intervention in both those with low and high step counts (p<0.05).

FIG. 4 plots the mean values (J) for the total work flexor at baseline and at the end of the study (V7) for each step-count (all values were divided by subject's weight (in kg) at each visit). These values, together with the results in Table 1 above, show that total work flexor was significantly increased following the 6-month intervention in those with low step counts (p<0.05).

In summary, the main effects were on the flexion (strength in flex leg) and predominantly in low step-count volunteers. Some improvements were also seen after the nutritional intervention period alone (p<0.05).

The other isokinetic parameters tested (torque max extensor, total work extensor and ratio torque flexor/extensor) were found to be statistically non-significant for each level of step count.

Looking at the effect of the nutritional composition alone, however, the torque ratio (flexor/extensor) was significantly improved following 2 months of intervention in all subjects (p<0.05). This can be seen from Table 2 below as the change from baseline at visit 3 was statistically significant (p<0.05) for all quartiles.

TABLE 2
Mixed model results for the Isokinetic Parameters. Estimates, standard
errors and 95% CI for change from baseline at each visit are presented
for Q1, median and Q3 of baseline
(Full Analysis Set)

| | | Quartiles of | | | | | |
|---|---|---|---|---|---|---|---|
| | | Isokinetics at | | Std. | | | |
| Isokinetic Parameters | Visit | Baseline | Estimate | Error | 2.5% | 97.5% | P-value |
| Ratio Torque Flexor/Extensor | Visit 3-visit 1 | First Quartile | 0.28 | 0.04 | 0.19 | 0.36 | <.0001 |
| | | Median | 0.11 | 0.03 | 0.05 | 0.18 | 0.0009 |
| | | Third Quartile | −0.08 | 0.04 | −015 | −0.00 | 0.0481 |
| Torque Max Extensor (Nm) | Visit 3-visit 1 | First Quartile | 0.09 | 0.07 | −0.04 | 0.22 | 0.1799 |
| | | Median | −0.10 | 0.05 | −0.20 | −0.00 | 0.0406 |
| | | Third Quartile | −0.17 | 0.06 | −0.29 | −0.06 | 0.0030 |
| Total Work Extensor (J) | Visit 3-visit 1 | First Quartile | 0.45 | 0.24 | −0.02 | 0.93 | 0.0605 |
| | | Median | −0.25 | 0.19 | −0.63 | 0.13 | 0.1909 |
| | | Third Quartile | −0.64 | 0.21 | −1.07 | −0.22 | 0.0031 |
| Torque Max Flexor (Nm) | Visit 3-visit 1 | First Quartile | 0.09 | 0.03 | 0.03 | 0.14 | 0.0021 |
| | | Median | 0.03 | 0.02 | −0.01 | 0.07 | 0.1609 |
| | | Third Quartile | −0.01 | 0.02 | −0.05 | 0.04 | 0.7666 |
| Total Work Flexor (J) | Visit 3-visit 1 | First Quartile | 0.39 | 0.15 | 0.09 | 0.68 | 0.0099 |
| | | Median | 0.05 | 0.12 | −0.18 | 0.29 | 0.6563 |
| | | Third Quartile | −0.32 | 0.14 | −0.59 | −0.04 | 0.0238 |

Results: Secondary Outcomes

Balance Test

For the balance test, subjects were classified according to total recorded time of balance training (minutes). The following levels were considered:
Low balance training: 0<=Total time of balance training<=320
High balance training: Total time of balance training>=321

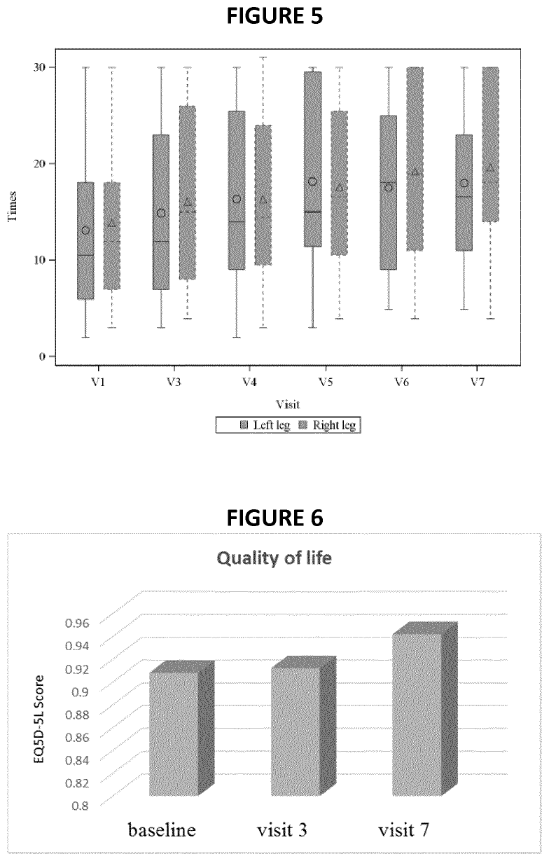

FIG. 5 is a boxplot of the balance test scores showing the balance times for the left leg and the right leg at baseline (V1), after the nutrition period (V3), at each month of the physical activity programme (V4, V5, V6) and at the end of the study (V7).

This figure shows that from the baseline to V7 there was an increase of approximately 36% (left leg) and approximately 41% (right leg) in the standing time on one leg in both low and high balance training time. In other words, balance outcomes were improved in all subjects at the end of the intervention (p<0.001). This is consistent with the results for the isokinetic flexion parameters.

MMRM analysis (not shown) demonstrated that nutritional intervention alone led to an improvement in the lowest score groups at baseline (Q1 and Q2).

Knee Pain (VAS)

Knee pain in subjects was measured using the Visual Analog Scale (VAS). The Visual Analog Scale is a psychometric response scale from 0 to 10, where 0 is no pain ever, 2 is mild plan, 5-6 is moderate pain, 8-9 is severe pain and 10 is the worst pain.

MMRM analysis demonstrated that there was a significant decrease (p<0.01) in knee pain by approximately 42% at the end of the study in all subjects. This was already observed after nutrition alone in the people reporting higher pain score (Q2 and Q3).

Knee Injury and Osteoarthritis Outcome Score (KOOS)

KOOS is a questionnaire which is well known in the art for quantifying joint injury to osteoarthritis (see for example: Roos E M et al., Health Qual Life Outcomes 2003; 1:64).

The questionnaire consists of 5 subscales: pain, other symptoms, function in daily living (ADL), function in sport and recreation (Sport/Rec) and knee related Quality of Life (QOL). Standardized answer options are given, the previous week is the time period considered when answering the questions, each question is assigned a score from 0 to 4, and a normalized score (100 indicating no symptoms and 0 indicating extreme symptoms) is calculated for each subscale.

The volunteers were given the KOOS in this example, and the change from baseline to visit 7 for the scores is shown in Table 3 below along with the ANCOVA model analysis.

TABLE 3

| KOOS Score | | Estimate | Std. Error | 2.5% | 97.5% | P-value |
|---|---|---|---|---|---|---|
| KOOS Pain* | Low step count | 7.85 | 1.94 | 3.72 | 11.97 | 0.0010 |
| | Average step count | 11.01 | 2.32 | 6.05 | 15.96 | 0.0003 |
| | High step count | 11.43 | 1.96 | 7.25 | 15.61 | <.0001 |
| KOOS Symptoms | Low step count | 3.22 | 3.67 | −4.60 | 11.03 | 0.3940 |
| | Average step count | 0.22 | 4.57 | −9.51 | 9.96 | 0.9616 |
| | High step count | 0.70 | 3.51 | −6.78 | 8.19 | 0.8439 |
| KOOS ADL* | Low step count | 8.34 | 1.92 | 4.24 | 12.44 | 0.0006 |
| | Average step count | 8.12 | 2.46 | 2.87 | 13.37 | 0.0049 |
| | High step count | 13.00 | 1.98 | 8.79 | 17.21 | <.0001 |
| KOOS Sport* | Low step count | 8.26 | 2.15 | 3.68 | 12.84 | 0.0016 |
| | Average step count | 9.20 | 2.60 | 3.66 | 14.75 | 0.0030 |
| | High step count | 12.31 | 2.13 | 7.76 | 16.85 | <.0001 |
| KOOS QOL | Low step count | 27.32 | 5.43 | 15.74 | 38.91 | 0.0002 |
| | Average step count | 22.28 | 6.40 | 8.64 | 35.92 | 0.0033 |
| | High step count | 20.15 | 5.31 | 8.85 | 31.46 | 0.0017 |

The ANCOVA model will include baseline KOOS score as covariate and levels of Garmin compliance as fixed effect.

It can be seen from this data that the change from baseline at V7 was statistically significant for KOOS pain (approximately 5.5% decrease), ADL (approximately 4% decrease), and sport (approximately 5.5% decrease) at each level of step count (p<0.005).

There was also a highly significant improvement in the quality of life score for all step counts: approximately 51% improvement from baseline (p<0.005).

Quality of Life: EQSD-5L

To further assess the effect on quality of life, volunteers were asked to complete the EQ5D-5L questionnaire. The change from baseline for the EQ5D-5L scores is shown in FIG. 6. The change from baseline to visit 7 was statistically significant at Q1 (p<0.005).

Biomarkers CTxl, CTX2 and P1NP

CTxl and P1NP are biomarkers of bone turnover; CTX2 is a biomarker of cartilage breakdown. These were monitored in the volunteers and at 6 months each of the biomarkers appeared to be positively modulated by the combined programme of nutrition and exercise (p<0.01). In particular there was a decrease of about 22% in cartilage breakdown and a decrease of about 20% in bone breakdown. Some effects were also already observed after nutrition alone.

### CONCLUSION

This example demonstrates that improved mobility is achieved in healthy, ageing subjects with the composition of the present invention, and that this improvement is enhanced by combining this composition with physical activity.

This example also demonstrates that maintained or improved joint function and muscle strength is achieved in healthy, ageing subjects with the composition of the present invention, and that this effect is enhanced by combining this composition with physical activity.

This intervention programme particularly showed that a significant decrease in knee pain could be achieved (approximately 42% decrease in VAS) and that this led to an improved quality of life score (approximately 51% increase in KOOS-QoL) for participants.

All publications mentioned in the above specification are herein incorporated by reference. Various modifications and variations of the described compositions, uses and methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

1. A composition comprising:
about 1500 mg to about 9000 mg of glucosamine or a pharmaceutically-acceptable derivative thereof per 100 g of the composition;
about 300 mg to about 2000 mg of calcium per 100 g of the composition;
about 50 mg to about 150 mg of vitamin C per 100 g of the composition;
about 3 μg to about 30 μg of vitamin D per 100 g of the composition; and
about 5 mg to about 25 mg of zinc per 100 g of the composition.

2. The composition of claim 1, wherein the composition comprises:
about 2500 mg to about 8000 mg of glucosamine or a pharmaceutically-acceptable derivative thereof per 100 g of the composition;
about 300 mg to about 1700 mg of calcium per 100 g of the composition;
about 70 mg to about 110 mg of vitamin C per 100 g of the composition;
about 5 μg to about 16 μg of vitamin D per 100 g of the composition; and
about 10 mg to about 20 mg of zinc per 100 g of the composition.

3. The composition of claim 1 further comprising about 15 g to about 30 g of protein per 100 g of the composition.

4. The composition of claim 1, wherein the composition is in the form of a powder.

5. The composition of claim 1, wherein the composition comprises a milk powder.

6. The composition of claim 1 comprising:
about 3000 mg of glucosamine sulphate per 100 g of the composition;
about 1700 mg of calcium per 100 g of the composition;
about 70 mg of vitamin C per 100 g of the composition;
about 16 μg of vitamin D per 100 g of the composition;
about 13 mg of zinc per 100 g of the composition, and
about 22 g of protein per 100 g of the composition.

7. The composition of claim 1, wherein vitamin D is vitamin D₃.

8. The composition of claim 1, wherein the composition provides:
about 200 to about 500 Kcal per 100 g of the composition;
about 15 g to about 30 g of protein per 100 g of the composition;
about 5 g to about 25 g of fat per 100 g of the composition;
about 40 g to about 60 g of carbohydrate per 100 g of the composition; and
about 200 mg to about 400 mg of sodium per 100 g of the composition.

9. The composition of claim 1 in the form of a powdered food product.

10. A beverage product comprising a composition comprising:
about 1500 mg to about 9000 mg of glucosamine or a pharmaceutically-acceptable derivative thereof per 100 g of the composition;
about 300 mg to about 2000 mg of calcium per 100 g of the composition;
about 50 mg to about 150 mg of vitamin C per 100 g of the composition;
about 3 μg to about 30 μg of vitamin D per 100 g of the composition; and
about 5 mg to about 25 mg of zinc per 100 g of the composition in the form of a powder which has been reconstituted in a liquid.

11. (canceled)

12. A method for maintaining or improving joint function mobility, and muscle strength in an ageing, healthy subject comprising administering a composition comprising:
about 1500 mg to about 9000 mg of glucosamine or a pharmaceutically-acceptable derivative thereof per 100 g of the composition;
about 300 mg to about 2000 mg of calcium per 100 g of the composition;
about 50 mg to about 150 mg of vitamin C per 100 g of the composition;
about 3 μg to about 30 μg of vitamin D per 100 g of the composition; and
about 5 mg to about 25 mg of zinc per 100 g of the composition to the subject.

13. The method of claim 12, wherein the subject is aged over about 40 and/or is a pre-osteoarthritis subject.

14. The method of claim 12, wherein the method further comprises at least one physical activity.

15. (canceled)

16. The composition of claim 1, wherein the composition comprises
about 7500 mg of glucosamine sulphate per 100 g of the composition;
about 1350 mg of calcium per 100 g of the composition;
about 110 mg of vitamin C per 100 g of the composition;
about 7.5 μg of vitamin D per 100 g of the composition; and
about 20 mg of zinc per 100 g of the composition.
